(19) United States
(12) Patent Application Publication
(10) Pub. No.: US 2022/0280700 A1
(43) Pub. Date: Sep. 8, 2022

(54) Temperature-Sensitive Hydrogel Composition for Preventing Tissue Adhesion, and Preparation Method Therefor

(71) Applicant: REGEN BIOCHARM CO., LTD., Seongnam-si, Gyeonggi-do (KR)

(72) Inventors: Gyung Gyun KIM, Seoul (KR); Myung CHOI, Seoul (KR); Sang Jin KIM, Busan (KR)

(21) Appl. No.: 17/638,821

(22) PCT Filed: Aug. 31, 2020
(86) PCT No.: PCT/KR2020/011633

§ 371 Date: Feb. 26, 2022

(30) Foreign Application Priority Data
Aug. 30, 2019 (KR) 10-2019-0107356

(51) Int. Cl. A61L 31/04; A61L 31/14; A61L 31/16

(52) U.S. Cl. CPC A61L 31/041; A61L 31/145; A61L 31/16; A61L 2300/416

(57) ABSTRACT

The present invention relates to a hydrogel composition for preventing tissue adhesion, and a preparation method therefor, and the hydrogel composition is composed of a polyethylene oxide-polypropylene oxide-polyethylene oxide terpolymer, a water-insoluble hyaluronic acid, sodium alginate and purified water and is prepared through the preparation method comprising: a copolymer melting step of heating, for one to two hours at a temperature of 60-100° C., a polyethylene oxide-polypropylene oxide-polyethylene oxide terpolymer having a molecular weight of 1-500 kDa, and melting same; a hyaluronic acid mixing step of mixing a molten product, having been prepared through the copolymer melting step, with a water-insoluble hyaluronic acid and stirring same at a temperature of 10-20° C.; and a sodium alginate mixing step of mixing a mixture, having been prepared through the hyaluronic acid mixing step, with sodium alginate and stirring same at a temperature of 5-20° C. In a hydrogel composition for preventing tissue adhesion, which is composed of the ingredients and is prepared by the preparation method, and a preparation method therefor, a polyethylene oxide-polypropylene oxide-polyethylene oxide terpolymer having the capacity to suppress tissue adhesion is used as a basic structure, and a water-insoluble hyaluronic acid and sodium alginate are blended, and thus a hydrogel composition for preventing tissue adhesion, to be uniformly coated on an in vivo wound site, is provided.

### TECHNICAL FIELD

The present invention relates to a hydrogel composition for preventing tissue adhesion that has, as a basic skeleton, a polyethylene-oxide/polypropylene-oxide/polyethylene-oxide terpolymer having an effect of inhibiting tissue adhesion, and is evenly applied to a wound site in the body by blending the terpolymer with water-insoluble hyaluronic acid and sodium alginate, and a method of preparing the same.

### BACKGROUND ART

In general, adhesion between organs and tissues after surgery is a natural phenomenon that occurs in the process of proliferation and regeneration of cells in damaged tissue. However, strong tissue adhesion or unintentional adhesion with other tissues and organs causes continuous discomfort or dysfunction in patients. In addition, reoperation for separation of the adhered tissues or organs may be required, and the adhesion may be life-threatening. Such tissue adhesion may occur anywhere in the human body, and in particular, adhesion occurs with a frequency of 70 to 95% after invasive surgery. Adhesion after surgery is known to be caused by entry of foreign substances, inflammatory reactions due to infection, bleeding at the surgical site, blood clotting, and rupture of the serous membrane. As can be seen from the causes of adhesion, bleeding has a great influence on initiating adhesion, but there is still no anti-adhesion agent that exhibits both an effect of penetrating active ingredients and preventing adhesion because the viscosity is increased due to body temperature. Currently used materials for hemostatic agents include biological natural polymers and non- biological natural polymers including polysaccharides and the like. These materials are used alone or in combination to form a specific structure.

Specifically, U.S. Pat. No. 7,262,181 discloses a hemostatic material containing water-soluble cellulose ether derivatives such as methyl cellulose, ethyl cellulose, and hydroxyethyl cellulose, wherein the hemostatic material is in the form of a fiber, woven fabric, nonwoven fabric, sponge, film or the like. Hemostatic agents having the form of a hydrogel, fiber, foam, nonwoven fabric or the like, as described above, are difficult to quickly and accurately apply to the wound site, and it is difficult to properly exhibit the effects thereof due to the risk of infection upon contact with medical personnel during treatment.

Anti-adhesion membranes or films have great disadvantages in which tissue adhesion occurs frequently at suture sites since suturing with the surrounding tissue using a suture is required in order to prevent movement of the anti-adhesion film at the application site, and it is difficult to introduce the anti-adhesion film into a complicated or small application part or a conduit. In an attempt to overcome these disadvantages, gel-type carboxymethyl cellulose, dextran 70, Flowgel prepared from a polyethylene-oxide/polypropylene-oxide/polyethylene-oxide terpolymer, Adcon-L (Gliatech) based on polylactic acid, Intercoat based on hyaluronic acid, and spray-type Spraygel based on polyethylene oxide are commercially available.

However, a gel-type anti-adhesion agent is known to take about 7 days to heal the wound at the surgical site, and has a problem of having low efficacy as the anti-adhesion agent because it is easily decomposed and/or absorbed in the body (in an aqueous solution phase) before the wound heals (J. M. Becker, et al., presented at clinical congress of Am. College of Surgeons, New Orleans, Oct. 22 (1995)).

As described above, although a great deal of research is underway on the prevention of tissue adhesion after surgery, conventional anti-adhesion agents still exhibit the above problems, and it is difficult to exhibit desired effects using the same, despite the high cost thereof. Therefore, there is still need for novel anti-adhesive agents.

### DISCLOSURE

### Technical Problem

It is one object of the present invention to provide a hydrogel composition for preventing tissue adhesion that has, as a basic skeleton, a polyethylene-oxide/polypropylene-oxide/polyethylene-oxide terpolymer having an effect of inhibiting tissue adhesion, and is evenly applied to a wound site in the body by blending the terpolymer with water-insoluble hyaluronic acid and sodium alginate, and a method of preparing the same.

It is another object of the present invention to provide a hydrogel composition for preventing tissue adhesion that stably releases a sparingly soluble anticancer agent using the melting point of the polyethylene-oxide/polypropylene-oxide/polyethylene-oxide terpolymer and the repulsive force between the polymers, and a method of preparing the same.

It is another object of the present invention to provide a hydrogel composition for preventing tissue adhesion that contains a biodegradable polymer and thus exhibits excellent biodegradability, and a method of preparing the same.

### Technical Solution

The objects of the present invention are achieved by providing a hydrogel composition for preventing tissue adhesion containing a polyethylene-oxide/polypropylene-oxide/polyethylene-oxide terpolymer, water-insoluble hyaluronic acid, sodium alginate, and purified water.

In a preferred embodiment, the hydrogel composition for preventing tissue adhesion may contain to 30% by weight of the polyethylene-oxide/polypropylene-oxide/polyethylene-oxide terpolymer, 2.5 to 4.5% by weight of the water-insoluble hyaluronic acid, 0.1 to 1% by weight of the sodium alginate, and the balance of the purified water.

In another preferred embodiment, the polyethylene-oxide/polypropylene-oxide/polyethylene- oxide terpolymer may have a molecular weight of 1 to 500 kDa.

In another preferred embodiment, the water-insoluble hyaluronic acid may be prepared by mixing 95 to 99% by weight of an aqueous ethanol solution with 1 to 5% by weight of hyaluronic acid having a molecular weight of 500 to 3,000 kDa to prepare a mixture and further mixing the mixture with 0.02 to 0.1 parts by weight of a crosslinking agent based on 100 parts by weight of hyaluronic acid contained in the mixture.

In another preferred embodiment, the aqueous ethanol solution may have a pH of 9.5 to 13 and a mass concentration of 70 to 80%.

In another preferred embodiment, the crosslinking agent may include 1,4-butanediol diglycidyl ether.

In another preferred embodiment, the hydrogel composition for preventing tissue adhesion may further contain 0.1 to 10 parts by weight of a sparingly soluble anticancer agent based on 100 parts by weight of the hydrogel composition for preventing tissue adhesion, and the sparingly soluble anticancer agent may be selected from the group consisting of docetaxel, docetaxel hydrate, paclitaxel, paclitaxel hydrate, capecitabine, and capecitabine hydrate.

In another preferred embodiment, the hydrogel composition for preventing tissue adhesion may further contain 10 to 50 parts by weight of a biodegradable polymer based on 100 parts by weight of the polyethylene-oxide/polypropylene-oxide/polyethylene-oxide terpolymer contained in the hydrogel composition for preventing tissue adhesion, wherein the biodegradable polymer is selected from the group consisting of poly-L-lactic-acid (PLLA), poly-lactic-co-glycolic acid (PLGA), polydioxanone (PDO), and polycaprolactone (PCL).

The objects of the present invention are achieved by providing a method for preparing a hydrogel composition for preventing tissue adhesion, the method including a terpolymer-melting step of melting a polyethylene-oxide/polypropylene-oxide/polyethylene-oxide terpolymer having a molecular weight of 1 to 500 kDa by heating at a temperature of 60 to 100° C. for 1 to 2 hours, a hyaluronic-acid-mixing step including mixing the melted product prepared in the terpolymer-melting step with water-insoluble hyaluronic acid, followed by stirring at a temperature of 10 to 20° C., and a sodium-alginate-mixing step including mixing the mixture prepared in the hyaluronic-acid-mixing step with sodium alginate, followed by stirring at a temperature of 5 to 20° C.

In a preferred embodiment, the method may further include a sparingly-soluble-anticancer-agent-mixing step of mixing the melted product prepared in the terpolymer-melting step with a sparingly soluble anticancer agent between the terpolymer-melting step and the hyaluronic-acid-mixing step, wherein the sparingly soluble anticancer agent is selected from the group consisting of docetaxel, docetaxel hydrate, paclitaxel, paclitaxel hydrate, capecitabine, and capecitabine hydrate.

In another preferred embodiment, the method may further include a biodegradable-polymer-mixing step of mixing the melted product prepared in the terpolymer-melting step with a biodegradable polymer between the terpolymer-melting step and the hyaluronic-acid-mixing step, wherein the biodegradable polymer is selected from the group consisting of poly-L-lactic-acid (PLLA), poly-lactic-co-glycolic acid (PLGA), polydioxanone (PDO), and polycaprolactone (PCL).

### Advantageous Effects

In addition, the present invention has an excellent effect of providing a hydrogel composition for preventing tissue adhesion that has, as a basic skeleton, a polyethylene-oxide/polypropylene-oxide/polyethylene-oxide terpolymer having an effect of inhibiting tissue adhesion, and is thus capable of being evenly applied to a wound site in the body by blending the terpolymer with water-insoluble hyaluronic acid and sodium alginate, and a method of preparing the same.

In addition, the present invention has an excellent effect of providing a hydrogel composition for preventing tissue adhesion that stably releases a sparingly soluble anticancer agent using the melting point of the polyethylene-oxide/polypropylene-oxide/polyethylene-oxide terpolymer and the repulsive force between the polymers, and a method of preparing the same.

In addition, the present invention has an excellent effect of providing a hydrogel composition for preventing tissue adhesion that contains a biodegradable polymer and thus exhibits excellent biodegradability, and a method of preparing the same.

### DESCRIPTION OF DRAWINGS

FIG. 1 is a graph showing changes in the viscosity of a hydrogel composition for preventing tissue adhesion depending on the content of polyethylene-oxide/polypropylene-oxide/polyethylene-oxide terpolymer.

FIG. 2 is a graph showing the result of a comparative analysis regarding the anti-tumor effect upon drug administration, wherein Vehicle represents PBS alone, DTX 10 mpk represents docetaxel 10 mg, DTX-88 10mpk represents docetaxel 10 mg contained in the hydrogel composition for preventing tissue adhesion, and DTX-88 20mpk represents docetaxel 20 mg contained in the hydrogel composition for preventing tissue adhesion, and the result of observation of the tumor size change for 30 days after direct injection into the affected area is shown.

FIG. 3 shows the data regarding the size of the tumor measured after directly injecting the drug and hydrogel composition for preventing tissue adhesion into the incised affected site, wherein Vehicle represents PBS alone, DTX 10 mpk represents docetaxel 10 mg, DTX-88 10mpk represents docetaxel 10 mg contained in the hydrogel composition for preventing tissue adhesion, and DTX-88 20mpk represents docetaxel 20 mg contained in the hydrogel composition for preventing tissue adhesion.

FIG. 4 shows the data regarding the size of the tumor measured 30 days after directly injecting the drug and hydrogel composition for preventing tissue adhesion into the incised affected site, wherein Vehicle represents PBS alone, DTX 10mpk represents docetaxel 10 mg, DTX-88 10mpk represents docetaxel 10 mg contained in the hydrogel composition for preventing tissue adhesion, and DTX-88 20mpk represents docetaxel 20 mg contained in the hydrogel composition for preventing tissue adhesion.

FIG. 5 is an image showing a subject when only the Vehicle (PBS) of FIG. 4 is injected into the affected area.

FIG. 6 is an image showing a subject when the DTX-88 10mpk of FIG. 4 is injected into the affected area.

FIG. 7 shows data indicating whether or not adhesion of surrounding organs occurs after incision of the affected area in FIG. 4.

FIG. 8 is an image showing a subject when the DTX-88 20mpk of FIG. 4 is injected into the affected area.

FIG. 9 shows (a) data obtained by measuring the average particle size of the biodegradable polymer (PLLA) with a particle size analyzer when the polyethylene-oxide/polypropylene-oxide/polyethylene-oxide terpolymer is present in an amount of 15% by weight, (b) data obtained by measuring the average particle size of the biodegradable polymer (PLLA) with a particle size analyzer when the polyethylene-oxide/polypropylene-oxide/polyethylene-oxide terpolymer is present in an amount of 20% by weight, and (c) data obtained by measuring the average particle size of the biodegradable polymer (PLLA) with a particle size analyzer when the polyethylene-oxide/polypropylene-oxide/polyethylene-oxide terpolymer is present in an amount of 25% by weight.

FIG. 10 shows (a) an image of the result of observation with an optical microscope with respect to a hydrogel composition for preventing tissue adhesion containing 20% by weight of the polyethylene-oxide/polypropylene-oxide/polyethylene-oxide terpolymer and PLLA, which is a biodegradable polymer, (b) an image of the result of observation with an optical microscope with respect to a hydrogel composition for preventing tissue adhesion containing 20% by weight of the polyethylene-oxide/polypropylene-oxide/polyethylene-oxide terpolymer and PLGA, which is a biodegradable polymer, (c) an image of the result of observation with an optical microscope with respect to a hydrogel composition for preventing tissue adhesion containing 20% by weight of the polyethylene-oxide/polypropylene-oxide/polyethylene-oxide terpolymer and PDO, which is a biodegradable polymer, and (d) an image of the result of observation with an optical microscope with respect to a hydrogel composition for preventing tissue adhesion containing 20% by weight of polyethylene-oxide/polypropylene-oxide/polyethylene-oxide and PCL, which is a biodegradable polymer.

### BEST MODE

Hereinafter, preferred embodiments of the present invention and the physical properties of each component will be described in detail such that they can be easily implemented by those skilled in the art, but should not be construed as limiting the technical sprit and scope of the present invention.

The hydrogel composition for preventing tissue adhesion according to the present invention contains a polyethylene-oxide/polypropylene-oxide/polyethylene-oxide terpolymer, water-insoluble hyaluronic acid, sodium alginate and purified water, wherein, preferably, the polyethylene-oxide/polypropylene-oxide/polyethylene-oxide terpolymer is present in an amount of 15 to 30% by weight, the water-insoluble hyaluronic acid is present in an amount of 2.5 to 4.5% by weight, the sodium alginate is present in an amount of 0.1 to 1% by weight, and the purified water is present in an amount corresponding to the balance.

The polyethylene-oxide/polypropylene-oxide/polyethylene-oxide terpolymer is present in an amount of 15 to 30% by weight, has a molecular weight of to 500 kDa, is the main ingredient of the hydrogel composition for preventing tissue adhesion according to the present invention, and serves to impart biocompatibility and an excellent anti-tissue adhesion effect to the hydrogel composition.

Also, the melting point of the polyethylene-oxide/polypropylene-oxide/polyethylene-oxide terpolymer and the repulsive force between the polymers constituting the terpolymer enable nanoization of a sparingly soluble anticancer agent so as to provide a hydrogel composition for preventing tissue adhesion that stably releases the sparingly soluble anticancer agent.

Also, the melting point of the polyethylene-oxide/polypropylene-oxide/polyethylene-oxide terpolymer and the repulsive force between the polymers constituting the terpolymer enable the biodegradable polymer to be granulated in the absence of a solvent to thus provide a hydrogel composition for preventing tissue adhesion that exhibits excellent biodegradability.

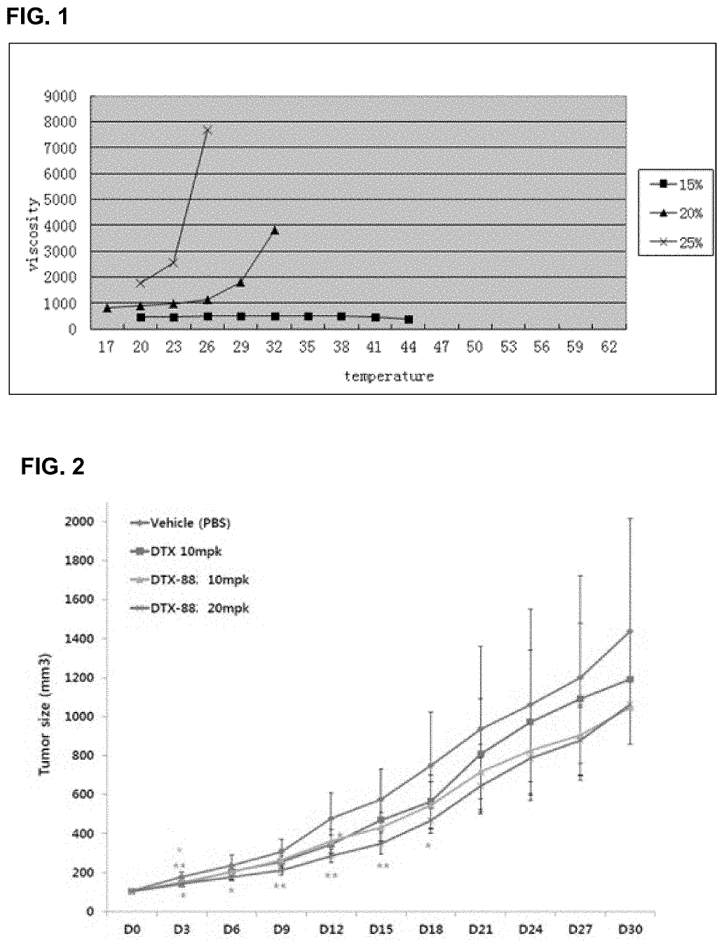

In this case, when the content of the polyethylene-oxide/polypropylene-oxide/polyethylene-oxide terpolymer is less than 15% by weight or is higher than 30% by weight, sol-gel action is not induced during heating, as shown in FIG. 1 below.

The water-insoluble hyaluronic acid is present in an amount of 2.5 to 4.5% by weight, exhibits skin moisturizing, skin regeneration and antibacterial effects, and serves to improve the morphological stability of the hydrogel composition for preventing tissue adhesion according to the present invention and to provide hemostatic activity due to the water-insolubility thereof.

The water-insoluble hyaluronic acid is prepared by mixing 1 to 5% by weight of hyaluronic acid having a molecular weight of 500 to 3,000 kDa with 95 to 99% by weight of an aqueous ethanol solution to prepare a mixture and further mixing the mixture with 0.02 to 0.1 parts by weight of a crosslinking agent based on 100 parts by weight of the hyaluronic acid contained in the mixture.

In this case, it is preferable to use sodium hyaluronate as the hyaluronic acid, and it is preferable to use an ethanol aqueous solution having a pH of 9.5 to 13 and a mass concentration of 70 to 80%.

When the mass concentration of the aqueous ethanol solution is less than 70%, aggregation of hyaluronic acid occurs, and when the mass concentration of the aqueous ethanol solution is higher than 80%, the viscosity of the hyaluronic acid increases excessively in the process of removing the ethanol component, so part of the crosslinking agent reacts rapidly, and thus a uniform viscosity cannot be obtained.

In this case, in the process of preparing the water-insoluble hyaluronic acid, it is preferable to apply a negative pressure of 0.1 to 1 atm, maintain a stirring speed of 50 to 100 rpm, and use 1,4-butanediol diglycidyl ether as the crosslinking agent.

When the content of the crosslinking agent is less than 0.02 parts by weight, the degree of crosslinking is lowered, and when the content of the crosslinking agent is higher than 0.1 parts by weight, partial gelation occurs in the solution, so homogenous mixing is not achieved.

After the solution becomes transparent during stirring under the above conditions, it is allowed to stand at 40 to 60° C. for 24 hours while the stirring is ceased, the negative pressure is removed and the temperature outside the reactor is maintained, and then the complete entire solution is dialyzed using a 7±2 kDa dialysis membrane for 2 to 3 days to complete the preparation.

The sodium alginate is present in an amount of 0.1 to 1% by weight, and imparts an anti-adhesion effect as well as improved viscosity and adhesiveness to the hydrogel composition for preventing tissue adhesion according to the present invention, thereby enabling the composition to be evenly applied to body wounds.

In this case, it is preferable to use sodium alginate, having a molecular weight of 300 to 1000 kDa.

In addition, when the content of sodium alginate is less than 0.1% by weight, the anti-adhesion effect cannot be achieved, and when the content of sodium alginate is higher than 1% by weight, the viscosity of the hydrogel composition for preventing tissue adhesion according to the present invention is excessively increased, and the lower critical solution temperature (LCST) thereof is increased.

Mixing with the sodium alginate is preferably performed at a constant stirrer temperature of 5 to 20° C., more preferably 10 to 15° C. When the temperature of the stirrer is less than 5° C., the water contained in the mixture may be partially frozen, and when the temperature of the stirrer is higher than 20° C., the viscosity of the mixture rises sharply, thus causing bubbles and heterogeneous mixing.

In addition, the hydrogel composition for preventing tissue adhesion may further contain 0.1 to 10 parts by weight of a sparingly soluble anticancer agent based on 100 parts by weight of the hydrogel composition for preventing tissue adhesion, the sparingly soluble anticancer agent may be selected from the group consisting of docetaxel, docetaxel hydrate, paclitaxel, paclitaxel hydrate, capecitabine, and capecitabine hydrate, and may serve to provide a hydrogel composition that has an effect of preventing tissue adhesion and an excellent anticancer effect by continuously releasing the anticancer agent into a surgical site immediately after various kinds of cancer surgery.

In addition, the hydrogel composition for preventing tissue adhesion contains 10 to 50 parts by weight of a biodegradable polymer based on 100 parts by weight of the polyethylene-oxide/polypropylene-oxide/polyethylene-oxide terpolymer contained in the hydrogel composition for preventing tissue adhesion, and the biodegradation polymer is selected from the group consisting of poly-L-lactic-acid (PLLA), poly-lactic-co-glycolic acid (PLGA), polydioxanone (PDO), and polycaprolactone (PCL). When the biodegradable polymer constituting the ingredient is contained, a hydrogel composition for preventing tissue adhesion having excellent biodegradability is provided.

In this case, when the content of the biodegradable polymer is less than 10 parts by weight, the effect is insufficient, and when the content of the biodegradable polymer is higher than 50 parts by weight, the morphological stability of the hydrogel composition for preventing tissue adhesion according to the present invention may be deteriorated.

In addition, the method for preparing a hydrogel composition for preventing tissue adhesion according to the present invention includes a terpolymer-melting step of melting a polyethylene-oxide/polypropylene-oxide/polyethylene-oxide terpolymer having a molecular weight of 1 to 500 kDa by heating at a temperature of 60 to 100° C. for 1 to 2 hours, a hyaluronic-acid-mixing step including mixing the melted product prepared in the terpolymer-melting step with water-insoluble hyaluronic acid, followed by stirring at a temperature of 10 to 20° C., and a sodium-alginate-mixing step including mixing the mixture prepared in the hyaluronic-acid-mixing step with sodium alginate, followed by stirring at a temperature of 5 to 20° C.

The terpolymer-melting step may be performed by heating the polyethylene-oxide/polypropylene- oxide/polyethylene-oxide terpolymer having a molecular weight of 1 to 500 kDa at a temperature of 60 to 100° C. for 1 to 2 hours. The polyethylene-oxide/polypropylene- oxide/polyethylene-oxide terpolymer is preferably used in an amount of 15 to 30% by weight with respect to the weight of the hydrogel composition for preventing tissue adhesion according to the present invention. When the content of the terpolymer is less than 15% by weight or higher than 30% by weight, the sol-gel action is not induced in a heated state, as shown in FIG. 1 below.

The hyaluronic-acid-mixing step includes mixing the melted product prepared in the terpolymer-melting step with water-insoluble hyaluronic acid, followed by stirring at a temperature of 10 to 20° C. The content of the water-insoluble hyaluronic acid that is mixed therein is preferably 2.5 to 4.5% by weight with respect to the total weight of the hydrogel composition for preventing tissue adhesion according to the present invention.

In this case, the process of preparing the water-insoluble hyaluronic acid is the same as that described with regard to the hydrogel composition for preventing tissue adhesion, and thus a description thereof will be omitted.

The sodium-alginate-mixing step includes mixing the mixture prepared in the hyaluronic-acid-mixing step with sodium alginate, followed by stirring at a temperature of 5 to 20° C. Preferably, the sodium alginate has a molecular weight of 300 to 1,000 kDa.

In addition, the amount of sodium alginate that is mixed in the sodium-alginate-mixing step is preferably 0.1 to 1% by weight with respect to the total weight of the hydrogel composition for preventing tissue adhesion according to the present invention. When the content of sodium alginate is less than 0.1% by weight, the anti-adhesion effect cannot be achieved, and when the content of sodium alginate is higher than 1% by weight, the viscosity of the hydrogel composition for preventing tissue adhesion according to the present invention is excessively increased, and the lower critical solution temperature (LCST) is increased.

When the stirring temperature in the sodium-alginate-mixing step is less than 5° C., the water contained in the mixture may be partially frozen, and when the stirring temperature is higher than 20° C., the viscosity of the mixture rises sharply, thus causing bubbles and heterogeneous mixing.

In addition, the method may further include a sparingly-soluble-anticancer-agent-mixing step of mixing the melted product prepared in the terpolymer-melting step with a sparingly soluble anticancer agent between the terpolymer-melting step and the hyaluronic-acid-mixing step, and the sparingly soluble anticancer agent is preferably selected from the group consisting of docetaxel, docetaxel hydrate, paclitaxel, paclitaxel hydrate, capecitabine, and capecitabine hydrate.

In this case, the content and role of the sparingly soluble anticancer agent used in the sparingly-soluble-anticancer-agent-mixing step are the same as those described with respect to the hydrogel composition for preventing tissue adhesion, and thus a description thereof will be omitted.

In addition, the method may further include a biodegradable-polymer-mixing step of mixing the melted product prepared in the terpolymer-melting step with a biodegradable polymer between the terpolymer-melting step and the hyaluronic-acid-mixing step, and the biodegradable polymer is preferably selected from the group consisting of poly-L-lactic-acid (PLLA), poly-lactic-co-glycolic acid (PLGA), polydioxanone (PDO), and polycaprolactone (PCL).

At this time, the content and role of the biodegradable polymer used in the biodegradable-polymer-mixing step are the same as those described with respect to the hydrogel composition for preventing tissue adhesion, and thus a description thereof will be omitted.

Hereinafter, the method for preparing the hydrogel composition for preventing tissue adhesion according to the present invention and the physical properties of the hydrogel composition for preventing tissue adhesion prepared using the method will be described with reference to Examples.

TABLE 1
Preparation of hydrogel composition for preventing tissue adhesion

| | | Water-insoluble | Sodium |
|---|---|---|---|
| Item | Terpolymer | hyaluronic acid | alginate |
| Example 1 | 14 | 0.9 | 0.5 |
| Example 2 | | 1.0 | |
| Example 3 | | 3.0 | |
| Example 4 | | 3.1 | |
| Example 5 | 15 | 0.9 | |
| Example 6 | | 1.0 | |
| Example 7 | | 3.0 | |
| Example 8 | | 3.1 | |
| Example 9 | 25 | 0.9 | |
| Example 10 | | 1.0 | |
| Example 11 | | 3.0 | |
| Example 12 | | 3.1 | |
| Example 13 | 26 | 0.9 | |
| Example 14 | | 1.0 | |
| Example 15 | | 3.0 | |
| Example 16 | | 3.1 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 2
Preparation of docetaxel-containing hydrogel
composition for preventing tissue adhesion

| | | Water-insoluble | |
|---|---|---|---|
| Item | Docetaxel | hyaluronic acid | Terpolymer |
| Example 17 | 0.1 | 0.9 | 15 |
| Example 18 | | 1.0 | |
| Example 19 | | 3.0 | |
| Example 20 | | 3.1 | |
| Example 21 | 0.5 | 0.9 | |
| Example 22 | | 1.0 | |
| Example 23 | | 3.0 | |
| Example 24 | | 3.1 | |
| Example 25 | 1 | 0.9 | |
| Example 26 | | 1.0 | |
| Example 27 | | 3.0 | |
| Example 28 | | 3.1 | |
| Example 29 | 1.5 | 0.9 | |
| Example 30 | | 1.0 | |
| Example 31 | | 3.0 | |
| Example 32 | | 3.1 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 3
Preparation of paclitaxel-containing hydrogel
composition for preventing tissue adhesion

| | | Water-insoluble | |
|---|---|---|---|
| Item | Paclitaxel | hyaluronic acid | Terpolymer |
| Example 33 | 0.1 | 0.9 | 15 |
| Example 34 | | 1.0 | |
| Example 35 | | 3.0 | |
| Example 36 | | 3.1 | |
| Example 37 | 0.5 | 0.9 | |
| Example 38 | | 1.0 | |
| Example 39 | | 3.0 | |
| Example 40 | | 3.1 | |
| Example 41 | 1 | 0.9 | |
| Example 42 | | 1.0 | |
| Example 43 | | 3.0 | |
| Example 44 | | 3.1 | |
| Example 45 | 1.5 | 0.9 | |
| Example 46 | | 1.0 | |
| Example 47 | | 3.0 | |
| Example 48 | | 3.1 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 4
Preparation of capecitabine-containing hydrogel
composition for preventing tissue adhesion

| | | Water-insoluble | |
|---|---|---|---|
| Item | Capecitabine | hyaluronic acid | Terpolymer |
| Example 49 | 0.1 | 0.9 | 15 |
| Example 50 | | 1.0 | |
| Example 51 | | 3.0 | |
| Example 52 | | 3.1 | |
| Example 53 | 0.5 | 0.9 | |
| Example 54 | | 1.0 | |
| Example 55 | | 3.0 | |
| Example 56 | | 3.1 | |
| Example 57 | 1 | 0.9 | |
| Example 58 | | 1.0 | |
| Example 59 | | 3.0 | |
| Example 60 | | 3.1 | |
| Example 61 | 1.5 | 0.9 | |
| Example 62 | | 1.0 | |
| Example 63 | | 3.0 | |
| Example 64 | | 3.1 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 5
Preparation of PLLA-containing hydrogel
composition for preventing tissue adhesion
The terpolymer was first melted, and PLLA, which is a biodegradable
polymer, was added thereto near a glass transition temperature of PLLA,
followed by stirring for 30 minutes to prepare the title composition.

| | | | Water-insoluble |
|---|---|---|---|
| Item | Terpolymer | PLLA | hyaluronic acid |
| Example 65 | 15 | 1.4 | 1.0 |
| Example 66 | | 1.5 | |
| Example 67 | | 7.5 | |
| Example 68 | | 7.6 | |
| Example 69 | 20 | 1.9 | |
| Example 70 | | 2.0 | |
| Example 71 | | 10.0 | |
| Example 72 | | 10.1 | |
| Example 73 | 25 | 2.4 | |
| Example 74 | | 2.5 | |
| Example 75 | | 12.5 | |
| Example 76 | | 12.6 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 6
Preparation of PLGA-containing hydrogel
composition for preventing tissue adhesion
The terpolymer was first melted, and PLGA, which is a biodegradable
polymer, was added thereto near a glass transition temperature of PLGA,
followed by stirring for 30 minutes to prepare the title composition.

| | | | Water-insoluble |
|---|---|---|---|
| Item | Terpolymer | PLGA | hyaluronic acid |
| Example 77 | 15 | 1.4 | 1.0 |
| Example 78 | | 1.5 | |
| Example 79 | | 7.5 | |
| Example 80 | | 7.6 | |
| Example 81 | 20 | 1.9 | |
| Example 82 | | 2.0 | |
| Example 83 | | 10.0 | |
| Example 84 | | 10.1 | |
| Example 85 | 25 | 2.4 | |
| Example 86 | | 2.5 | |
| Example 87 | | 12.5 | |
| Example 88 | | 12.6 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 7
Preparation of PDO-containing hydrogel
composition for preventing tissue adhesion
The terpolymer was first melted, and PDO, which is a biodegradable
polymer, was added thereto near the glass transition temperature of PDO,
followed by stirring for 30 minutes to prepare the title composition.

| | | | Water-insoluble |
|---|---|---|---|
| Item | Terpolymer | PDO | hyaluronic acid |
| Example 89 | 15 | 1.4 | 1.0 |
| Example 90 | | 1.5 | |
| Example 91 | | 7.5 | |
| Example 92 | | 7.6 | |
| Example 93 | 20 | 1.9 | |
| Example 94 | | 2.0 | |
| Example 95 | | 10.0 | |
| Example 96 | | 10.1 | |
| Example 97 | 25 | 2.4 | |
| Example 98 | | 2.5 | |
| Example 99 | | 12.5 | |
| Example 100 | | 12.6 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

TABLE 8
Preparation of PCL-containing hydrogel
composition for preventing tissue adhesion
The terpolymer was first melted, and PCL, which is a biodegradable
polymer, was added thereto near a glass transition temperature of PCL,
followed by stirring for 30 minutes to prepare the title composition.

| | | | Water-insoluble |
|---|---|---|---|
| Item | Terpolymer | PCL | hyaluronic acid |
| Example 101 | 15 | 1.4 | 1.0 |
| Example 102 | | 1.5 | |
| Example 103 | | 7.5 | |
| Example 104 | | 7.6 | |
| Example 105 | 20 | 1.9 | |
| Example 106 | | 2.0 | |
| Example 107 | | 10.0 | |
| Example 108 | | 10.1 | |
| Example 109 | 25 | 2.4 | |
| Example 110 | | 2.5 | |
| Example 111 | | 12.5 | |
| Example 112 | | 12.6 | |

Content percentage means weight percentage (%) in final aqueous solution
Terpolymer: polyethylene-oxide/polypropylene-oxide/polyethylene-oxide terpolymer having molecular weight of 1,000 to 500,000 g/mol
Water-insoluble hyaluronic acid: water-insoluble hyaluronic acid cross-linked with 70% aqueous ethanol solution
Water is present in the balance excluding the content of ingredients constituting the blend.

### <Example 113>Preparation of PLLA, PLGA, PDO and PCL particles

In Examples 65 to 112, a biodegradable polymer was added in a predetermined amount to the terpolymer, granulated, washed at least 3 times with distilled water without adding a water-insoluble hydrogel thereto, frozen at −70° C. or less and then lyophilized at −45° C. and 1 m bar to prepare particles.

### Experimental Example 1

The uniformity of the hydrogel compositions for preventing tissue adhesion prepared in Examples 1 to 16 was visually observed, and the result is shown in Table 9 below.

TABLE 9

| Example | 1 | 2 | 3 | 4 | 5 | 6 | 7 | 8 |
|---|---|---|---|---|---|---|---|---|
| State | X | ▴ | ▴ | X | ▴ | ⊚ | ⊚ | ▴ |
| Example | 9 | 10 | 11 | 12 | 13 | 14 | 15 | 16 |
| State | ▴ | ⊚ | ⊚ | ▴ | X | ▴ | ▴ | X |

⊚: Solution state is good
▴: Gelation partially occurs
X: Gelation entirely occurs

As shown in Table 9, when the content of the terpolymer is less than 15% by weight or is higher than 30% by weight, the morphology of the hydrogel composition is not homogenous and when the concentration of water-insoluble hyaluronic acid is less than 2.5% by weight or is higher than 4.5% by weight, the hydrogel composition is not homogeneous.

Experimental Example 2>Animal Test to Detect Anti-Adhesion Efficacy of Composite Hydrogel—Visual Findings at Autopsy

Experimental Example 2 is performed to determine the effect of preventing tissue adhesion and the hemostatic effect upon local bleeding with respect to Examples 6, 7, 10, and 11. The experiment to determine the anti-adhesion effect based on visual observation at autopsy was performed, and the result is shown in Table 12 below.

After anesthesia, the surgical sites of animals were epilated and disinfected with povidone, and a 4 to 5 cm incision was formed along the midline of the abdominal cavity. The cecum was isolated, and an abrasive wound having a size of 1 cm×2 cm was formed using a bone burr, and a wound having the same size was formed on the peritoneal membrane opposite thereto. Tissue adhesion was induced by fixing three places about 1 cm away from the frictional injury site with sutures such that the two damaged surfaces came into contact with each other. Each test substance was injected thereto. Animals that survived until the test end date were euthanized through CO₂ gas inhalation, autopsy and visual pathological examination were performed, and the adhesion degree, adhesive strength, and adhesion area were recorded for each animal. Adhesion site tissue was immobilized in 10% neutral formalin for histological examination.

At this time, the criteria used for measurement of the anti-adhesion effect are shown in Tables 10 to 11 below.

TABLE 10
Classification depending on degree
of adhesion of adhesion surfaces

0: No adhesion
1: One thin and strong adhesion site
2: Two or more thin and strong adhesion sites
3: Thick dot-type adhesion sites
4: Thick plane-type adhesion sites
5: Very thick adhesion sites provided with blood vessels

TABLE 11
Classification of adhesion surfaces depending
on force required to overcome adhesion

1: Adhesion is overcome upon application of weak force
2: Adhesion is overcome upon application of medium force
3: Adhesion is overcome upon application of strong force
4: Adhesion is too strong to overcome

TABLE 12

| Item | Example 6 | Example 7 | Example 10 | Example 11 |
|---|---|---|---|---|
| K-score₁ | 1.00 ± 0.81 | 0.63 ± 0.35 | 0.60 ± 0.30 | 0.90 ± 0.75 |
| K-score₂ | 0.60 ± 0.84 | 0.40 ± 0.64 | 0.42 ± 0.53 | 0.65 ± 0.54 |

₁Adhesion grading scale (Knightly score)
₂Adhesion grading scale (Hooker score)

As can be seen from Table 12, the hydrogel compositions prepared in Examples 6 to 7 and Examples 10 to 11 of the present invention exhibited an excellent anti-adhesion effect.

The evaluation of adhesion by visual observation was performed in accordance with the method suggested by Vlahos et al. (Vlahos A., Yu P., Lucas C. E., Ledgerwood A. M. Effect of a composite membrane of chitosan and Poloxamer gel on post-operative adhesive interactions, The American Surgeon 2001, 67:15-21). Specifically, classification of the adhesion surface depending on the degree of adhesion (score: 0 to 5, Table 10), classification depending on the strength at which the two surfaces are detached from each other when the adhesion surface is separated by hand (score: 1 to 4, Table 11), and evaluation of the degree of adhesion based on measurement of the area of the adhesion site were performed. The results are shown in Table 12.

### <Experimental Example 3>Animal Experiment to Determine Anti-Adhesion and Drug-Release Effects of Composite Hydrogel

The composite hydrogels prepared in Examples 17 to 32 were used.

The experiment was performed as follows. Mice were acquired and then stabilized for one week. The MKN74 gastric cancer gastric cell line was subcutaneously injected. When the average tumor size reached 100 mm³, the mice were grouped and then administered with the drug composite hydrogel. When the tumor size reached an average of 100 mm³, the size of the tumor was measured every 3 days, and the mice were weighed. After injection of the composite hydrogel, whether or not there was an abnormality in each mouse were determined. After composite hydrogel injection was completed, the tumor was excised, and the weight thereof was measured. In addition, whether or not adhesion occurred in the organs of the excised area was observed. Six mice were used as test subjects. The laboratory temperature was 22±2° C., the relative humidity was 0±10%, and the feed used herein was Purina laboratory animal rat feed. All of the provided drinking water was R/O water, and water quality inspection was conducted twice a year. The microbiological test was performed using sentinel animals. The species and strains of the experimental animals were: mouse, Balb/c nude, SPF. The animals were 5-week-old females purchased from Central Laboratory Animal SLC (Japan). The cell line used in the experiment was MKN74 (gastric cancer cell line, Korea cell line bank) and was cultured under culture conditions of RPM1640 (Welgene) +10% FBS (ATCC).

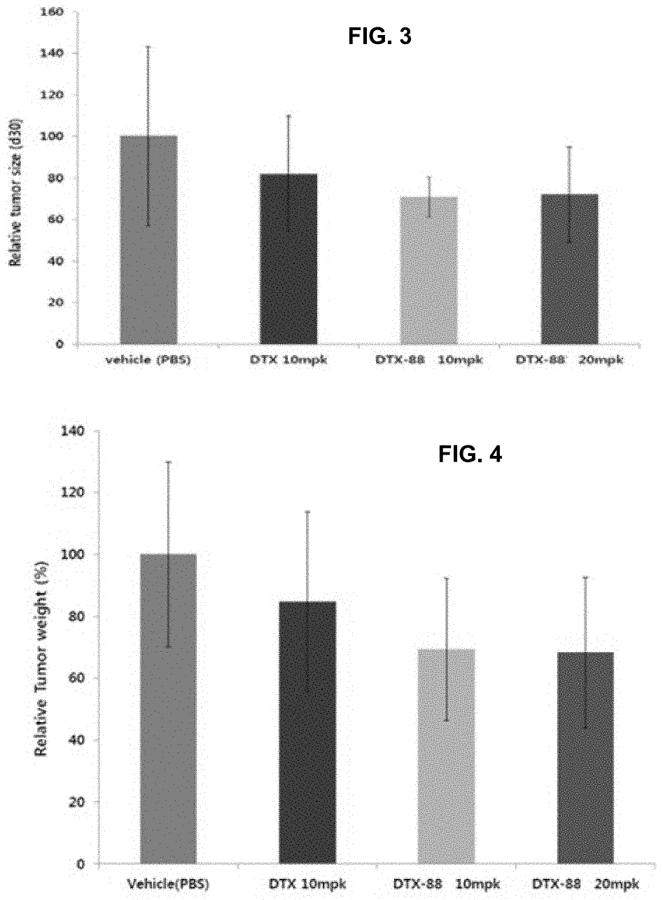

As can be seen from FIGS. 1 to 3 below, there was no significant difference between groups until the 6th day. However, it can be seen that from the 9th day, there was a difference in tumor size between the PBS-treated group and the group treated with the composite hydrogel containing the anticancer agent.

On the last (30^{th}) day, the tumor size of the composite hydrogel was reduced by 30% compared to the PBS-treated group.

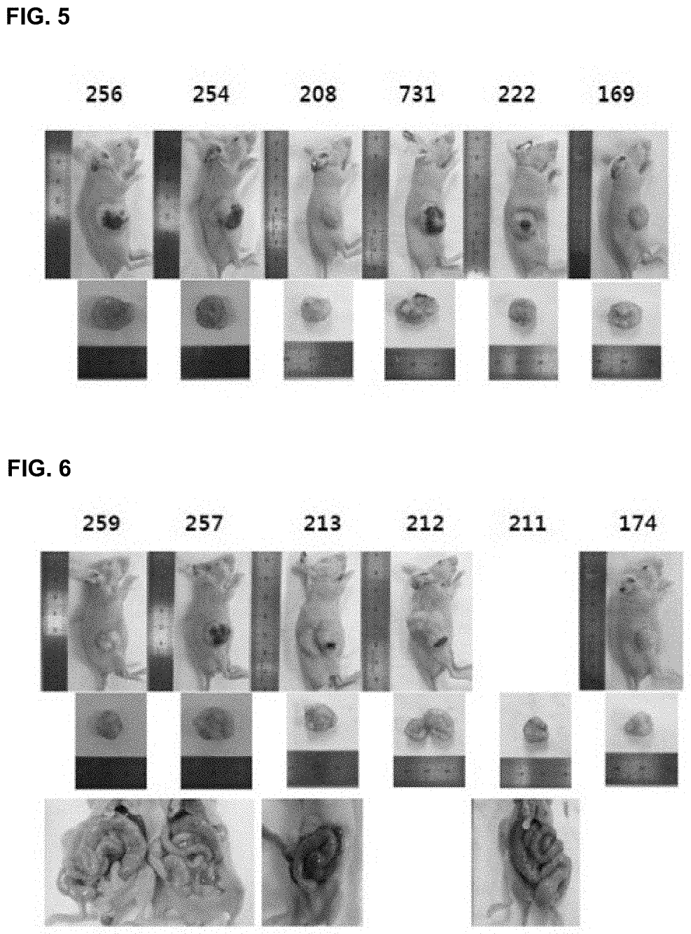

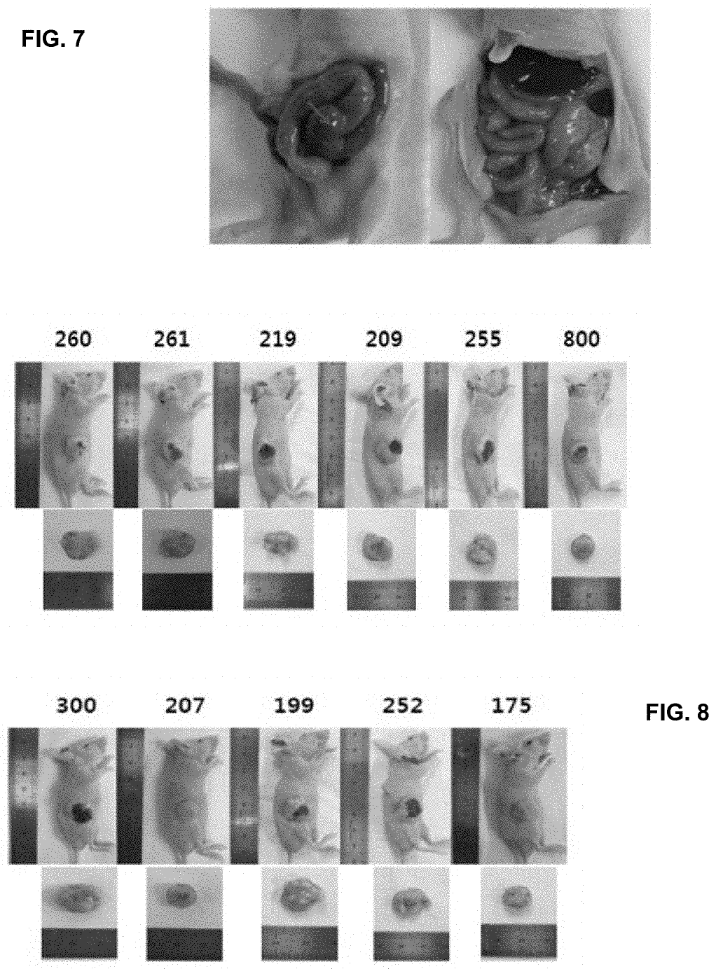

In addition, as can be seen from FIGS. 5 to 8, intestinal adhesion occurred in the PBS-treated group, whereas adhesion did not occur in the group treated with the hydrogel composition according to the present invention.

As a result, as can be seen from FIGS. 2 to 8, the tumor size was reduced by 30% in the group treated with the anticancer agent compared to the vehicle (PBS)-treated group. In addition, it can be seen that adhesion inside the organ did not occur after 30 days.

The hydrogel composition containing paclitaxel and capecitabine also showed results similar to those described above.

### <Experimental Example 4>Preparation of Biodegradable Particles During Preparation of Composite Hydrogel

The particles prepared in Example 113 were measured with a particle size analyzer. In addition, the morphology of the particles was observed at a magnification of 10× with a microscope.

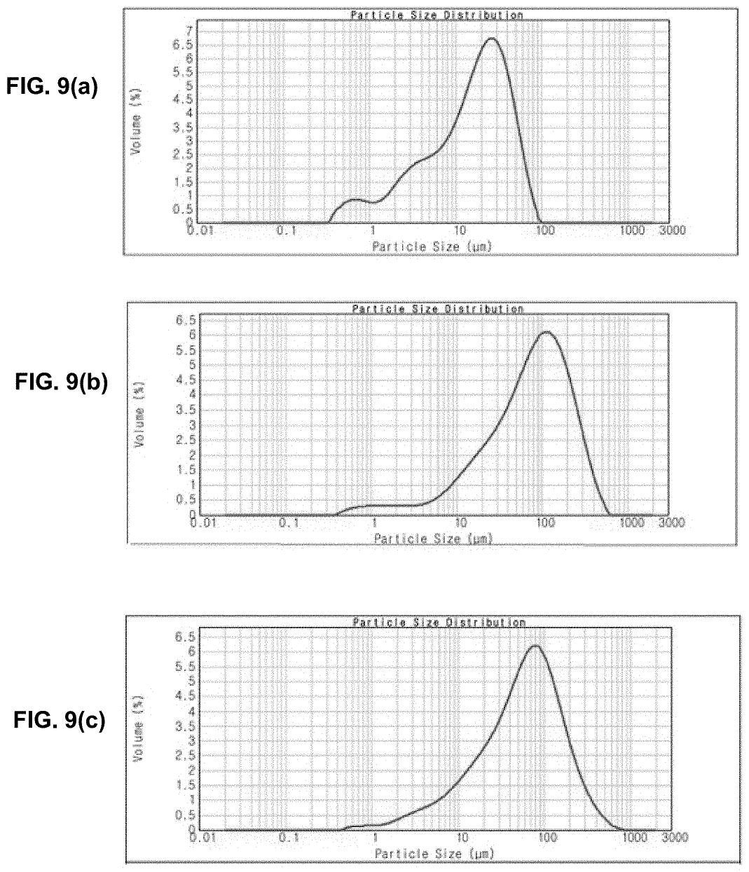

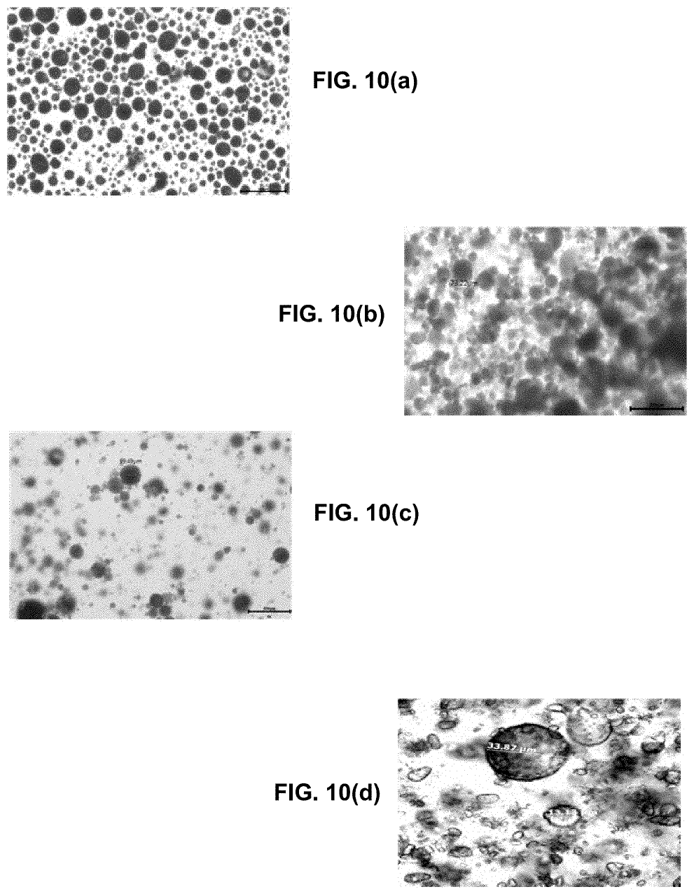

As can be seen from FIGS. 9 to 10, the particles that were prepared had a spherical shape and an average particle size of 10 to 100 micrometers.

However, it can be seen that the particle size decreased as the content of the terpolymer increased and the particle size increased as the content of the biodegradable polymer increased.

Therefore, the present invention provides a hydrogel composition for preventing tissue adhesion that has, as a basic skeleton, a polyethylene-oxide/polypropylene-oxide/polyethylene-oxide terpolymer having an effect of inhibiting tissue adhesion and is evenly applied to a wound site in the body by blending the terpolymer with water-insoluble hyaluronic acid and sodium alginate, and a method of preparing the same.

The present invention provides a hydrogel composition for preventing tissue adhesion that stably releases a sparingly soluble anticancer agent using the melting point of the polyethylene-oxide/polypropylene-oxide/polyethylene-oxide terpolymer and the repulsive force between the polymers, and a method of preparing the same.

The present invention provides a hydrogel composition for preventing tissue adhesion that contains a biodegradable polymer and thus exhibits excellent biodegradability, and a method of preparing the same.

1. A hydrogel composition for preventing tissue adhesion comprising:
15 to 30% by weight of a polyethylene-oxide/polypropylene-oxide/polyethylene-oxide terpolymer;
2.5 to 4.5% by weight of water-insoluble hyaluronic acid;
0.1 to 1% by weight of sodium alginate; and
the balance of purified water,
wherein the water-insoluble hyaluronic acid is prepared by mixing 95 to 99% by weight of an aqueous ethanol solution with 1 to 5% by weight of hyaluronic acid having a molecular weight of 500 to 3,000 kDa to prepare a mixture and further mixing the mixture with 0.02 to 0.1 parts by weight of a crosslinking agent based on 100 parts by weight of hyaluronic acid contained in the mixture.

2. The hydrogel composition according to claim 1, wherein the polyethylene-oxide/polypropylene-oxide/polyethylene-oxide terpolymer has a molecular weight of 1 to 500 kDa.

3. The hydrogel composition according to claim 1, wherein the aqueous ethanol solution has a pH of 9.5 to 13 and a mass concentration of 70 to 80%.

4. The hydrogel composition according to claim 1, wherein the crosslinking agent comprises 1,4-butanediol diglycidyl ether.

5. The hydrogel composition according to claim 1, further comprising 0.1 to 10 parts by weight of a sparingly soluble anticancer agent based on 100 parts by weight of the hydrogel composition for preventing tissue adhesion,
wherein the sparingly soluble anticancer agent is selected from the group consisting of docetaxel, docetaxel hydrate, paclitaxel, paclitaxel hydrate, capecitabine, and capecitabine hydrate.

6. The hydrogel composition according to claim 1, further comprising 10 to 50 parts by weight of a biodegradable polymer based on 100 parts by weight of the polyethylene-oxide/polypropylene-oxide/polyethylene-oxide terpolymer contained in the hydrogel composition for preventing tissue adhesion,
wherein the biodegradable polymer is selected from the group consisting of poly-L-lactic-acid (PLLA), poly-lactic-co-glycolic acid (PLGA), polydioxanone (PDO), and polycaprolactone (PCL).

7. A method for preparing a hydrogel composition for preventing tissue adhesion,
the method comprising:
a terpolymer-melting step of melting a polyethylene-oxide/polypropylene-oxide/polyethylene-oxide terpolymer having a molecular weight of 1 to 500 kDa by heating at a temperature of 60 to 100° C. for 1 to 2 hours;
a hyaluronic-acid-mixing step comprising mixing a melted product prepared in the terpolymer-melting step with water-insoluble hyaluronic acid, followed by stirring at a temperature of 10 to 20° C.; and
a sodium-alginate-mixing step comprising mixing a mixture obtained through the hyaluronic-acid-mixing step with sodium alginate, followed by stirring at a temperature of 5 to 20° C.

8. The method according to claim 7, further comprising a sparingly-soluble-anticancer-agent-mixing step of mixing the melted product prepared in the terpolymer-melting step with a sparingly soluble anticancer agent between the terpolymer-melting step and the hyaluronic-acid-mixing step,
wherein the sparingly soluble anticancer agent is selected from the group consisting of docetaxel, docetaxel hydrate, paclitaxel, paclitaxel hydrate, capecitabine, and capecitabine hydrate.

9. The method according to claim 7, further comprising a biodegradable-polymer-mixing step of mixing the melted product prepared in the terpolymer-melting step with a biodegradable polymer between the terpolymer-melting step and the hyaluronic-acid-mixing step,
wherein the biodegradable polymer is selected from the group consisting of poly-L-lactic-acid (PLLA), poly-lactic-co-glycolic acid (PLGA), polydioxanone (PDO), and polycaprolactone (PCL).
